SEQUENCE LISTING (6 sequences)

SEQ ID NO: 1 - DNA, 33 bp, Artificial
  source (1)..(33): oligonucleotide primer (sense)
ctacgtaccatggattttcaggtgcagattttc

SEQ ID NO: 2 - DNA, 24 bp, Artificial
  source (1)..(24): oligonucleotide primer (antisense)
ggttccagcagatctggatacggc

SEQ ID NO: 3 - DNA, 99 bp, Artificial
  source (1)..(99): oligonucleotide primer (sense)
tactggatcctcagctctgctatatcctggatgccatcctgtttctgtatggaattgtcctcaccctcctctactgtagagtgaagttcagcaggagcg

SEQ ID NO: 4 - DNA, 32 bp, Artificial
  source (1)..(32): oligonucleotide primer (antisense)
ctgctactcgaggattagcgagggggcagggc

SEQ ID NO: 5 - DNA, 101 bp, Artificial
  source (1)..(101): oligonucleotide primer (sense)
tactggatcccaaactctgctacctgctggatggaatcctcttcatctatggtgtcattctcactgccttgttcctgcgactgaagatccaagtgcgaaag

SEQ ID NO: 6 - DNA, 41 bp, Artificial
  source (1)..(41): oligonucleotide primer (antisense)
ctgctactcgaggactaaagctactgtggtggtttctcatg

(19) United States
(12) Patent Application Publication
(10) Pub. No.: US 2005/0118185 A1
(43) Pub. Date: Jun. 2, 2005

(54) Recombinant immunoreceptors

(75) Inventors: Andreas Hombach, Bruhl (DE); Hinrich Abken, Meudt (DE); Claudia Heuser, Weinheim (DE)

Correspondence Address: J. Mitchell Jones;MEDLEN & CARROLL, LLP, Suite 350, 101 Howard Street, San Francisco, CA, 94105, US

(73) Assignee: Cell Center Cologne GmbH, Koln (DE)

(21) Appl. No.: 10/870,636

(22) Filed: Jun. 17, 2004

Related U.S. Application Data
(60) Provisional application No. 60/479,149, filed on Jun. 18, 2003.

(51) Int. Cl. C07K 14/74; C07H 21/04; A61K 39/00

(52) U.S. Cl. 424/185.1; 435/69.1; 435/320.1; 435/325; 530/350; 536/23.2

(57) ABSTRACT

Described are recombinant immunoreceptors composed of an extracellular antigen binding domain (scFv) and a transmembrane region derived from the CD3ζ-chain and/or the FcεRIγ-chain linked to an intracellular signaling domain with cell activation properties. Moreover, nucleic acid sequences encoding said immunoreceptor, immune cells expressing said immunoreceptor as well as therapeutic uses of said immunoreceptor, e.g. adoptive immunotherapy, are described.

This application claims the benefit of U.S. provisional application 60/479,149, filed Jun. 18, 2003.

### FIELD OF THE INVENTION

The present invention relates to recombinant immunoreceptors composed of an extracellular antigen binding domain (scFv) and a transmembrane region and/or intracellular signaling domain derived from the CD3ζ-chain and/or the FcεRIγ-chain. In particular, the present invention relates to a recombinant immunoreceptor comprising (i) a first antigen binding segment comprising an scFv and (ii) a second segment linked to the first segment and comprising a transmembrane domain fused to an intracellular signaling domain, wherein (a) the transmembrane domain comprises an FcεRIγ-chain derived transmembrane domain and the intracellular signaling domain comprises a CD3ζ-chain derived intracellular signaling domain; (b) the transmembrane domain comprises a CD3ζ-chain derived transmembrane domain and the intracellular signaling domain comprises a CD3ζ-chain derived intracellular signaling domain; (c) the transmembrane domain comprises a CD3ζ-chain derived transmembrane domain and the intracellular signaling domain comprises an FcεRIγ-chain derived intracellular signaling domain; or (d) the transmembrane domain comprises a FcεRIγ-chain derived transmembrane domain and the intracellular signaling domain comprises an FcεRIγ-chain derived intracellular signaling domain. The present invention also relates to nucleic acid sequences encoding said immunoreceptor, cells expressing said immunoreceptor as well as therapeutic uses of said immunoreceptor.

### BACKGROUND OF THE INVENTION

The major pathway of the immune defense begins with the trapping of the antigen by accessory cells such as dendritic cells or macrophages. Upon specific recognition of the processed antigen, mature T helper cells can be triggered to become activated T helper cells. These activated T helper cells regulate both the humoral immune response by inducing the differentiation of b cells to antibody-producing plasma cells and control of cell-mediated immune response by activation of cytotoxic T lymphocytes (CTL) and natural killer cells.

T lymphocytes recognize antigen in the context of self MHC molecules by means of the T cell receptor (TCR). The TCR expressed on the surface of T cells is a disulfide-linked heterodimer non-covalently associated with an invariant structure, the CD3 complex. CD3 is assumed to be responsible for intracellular signaling following occupancy of the TCR by a ligand. The T cell receptor for antigen-CD3 complex (TCR/CD3) recognizes antigenic peptides that are presented to it by the MHC proteins. Complexes of MHC and peptide are expressed an the surface of antigen presenting cells and other T cell targets. Stimulation of the TCR/CD3 complex results in activation of the T cell and a consequent antigen-specific immune response.

Like the Ig, the TCRs are composed of variable segments (responsible for the specific recognition of the antigen) and constant regions (responsible for membrane anchoring and signal transduction). Two forms of T cell receptors for antigens are expressed on the surface of T cells. These contain either α/β heterodimers or γ/δ heterodimers. Accordingly, each of those chains is made of the V and C regions of the TCR, namely V_{α}C_{α}, V_{β}C_{β}, V_{γ}C_{γ}, and V_{δ}C_{δ}. T cells are capable of rearranging the genes that encode the α, β, γ and δ chains of the T cell receptor. T cell receptor gene rearrangements are analogous to those that produce functional immunoglobulins in B cells and the presence of multiple variable and joining regions in the genome allows the generation of T cell receptors with a diverse range of binding specificities. Each α/β or γ/δ heterodimer is expressed on the surface of the T cell in association with four invariant peptides. These are the γ, δ and ε subunits of the CD3 complex and the zeta (ζ) chain. The CD3 chains and the zeta subunit do not show variability, and are not involved directly in antigen recognition.

All the components of the T cell receptor are membrane proteins and consist of a leader sequence, externally-disposed N-terminal extracellular domains, a single membrane-spanning domain, and cytoplasmic tails. Most T cell receptor α/β heterodimers are covalently linked through disulphide bonds, but many γ/δ receptors associate with one another non-covalently. The zeta chain quantitatively forms either disulphide-linked ζ/η heterodimers or zeta-zeta homodimers.

Another example of a type of receptor on cells of the immune system is the Fc receptor. The interaction of antibody-antigen complexes with cells of the immune system results in a wide array of responses, ranging from effector functions such as antibody-dependent cytotoxicity, mast cell degranulation, and phagocytosis to immunomodulatory signals such as regulating lymphocyte proliferation, phagocytosis and target cell lysis. All these interactions are initiated through the binding of the Fc domain of antibodies or immune complexes to specialized cell surface receptors on hematopoietic cells. It is now well established that the diversity of cellular responses triggered by antibodies and immune complexes results from the structural heterogeneity of Fc receptors (FcRs). FcRs are defined by their specificity for immunoglobulin isotypes. Fc receptors for IgG are referred to as Fc_{γ}R, for IgE as Fc_{ε}R, for IgA as Fc_{α}R, etc. Structurally distinct receptors are distinguished by a Roman numeral, based an historical precedent. Structurally related although distinct genes within a group are denoted by A, B, and C.

Antigen-specific effector lymphocytes, such as tumor specific T cells (Tc), are very rare, individual-specific, limited in their recognition spectrum and difficult to obtain against most malignancies. Antibodies, on the other hand, are readily obtainable, more easily derived, have wider spectrum and are not individual-specific. The major problem of applying specific antibodies, e.g., for cancer immunotherapy lies in the inability of sufficient amounts of monoclonal antibodies (mAb) to reach large areas within solid tumors. In practice, many clinical attempts to recruit the humoral or cellular arms of the immune system for passive anti-tumor antibodies have not fulfilled expectations. While it has been possible to obtain anti-tumor antibodies, their therapeutic use has been limited so far to blood-borne tumors primarily because solid tumors are inaccessible to sufficient amounts of antibodies. The use of effector lymphocytes in adoptive immunotherapy, although effective in selected solid tumors, suffers on the other hand, from a lack of specificity or from the difficulty in recruiting tumor-infiltrating lymphocytes (TILs) and expanding such specific T cells for most malignancies. Previously, it has been tried to overcome these problems by use of recombinant immunoreceptors (Eshhar et al., Proc. Natl. Acad. Sci. USA 90 (1993), 720-724). Upon specific binding to antigen, these recombinant immunoreceptors with antibody-like specificity direct both CD4⁺ and CD8⁺ T cells to highly efficient, MHC-molecule independent cellular activation against antigen expressing target cells (Hombach et al., J. Immunol. 167 (2001), 1090-1096). The antigen binding domain of these receptors consists of an antibody derived single-chain fragment (scFv). However, the recombinant immunoreceptors described so far are still characterized by many disadvantages, e.g., result in low immunoreceptor mediated T cell activation against tumor cells.

Thus, what is needed to solve this technical problem is to provide immunoreceptors, e.g. for adoptive immunotherapy, that overcome the disadvantages of the immunoreceptors of the prior art.

### SUMMARY OF THE INVENTION

The solution to said technical problem is achieved by providing the embodiments characterized in the claims. For solving the above identified technical problem, a panel of recombinant immunoreceptors was generated that consist extracellularly of the same antigen binding and spacer domain whereas the transmembrane region and/or intracellular domain is derived from the CD3ζ and/or FcεRIγ. After expression of these γ- and ζ-chain immunoreceptors, (i) the stability of receptor expression in the presence and absence of the endogenous TCR, (ii) the influence of recombinant γ- or ζ-chain immunoreceptors expression on the stability and function of the endogenous CD3/TCR complex, (iii) the stability of recombinant receptor expression in peripheral blood T cells and (iv) receptor mediated cellular activation by γ- and ζ-chain immunoreceptors at different time points after receptor engraftment was recorded. The panel of recombinant ζ- and γ-chain immunoreceptors was expressed in mouse T cell lines, that either do not express endogenous CD3; or are defective in TCRα expression, and in human peripheral blood T cells, respectively. After expression in T cells that lack expression of endogenous CD3ζ the recombinant ζ receptor restored cell surface expression and function of the endogenous CD3/TCR complex whereas the homologous y receptor did not. In contrast, the presence of an endogenous TCR substantially impaired the stability of ζ-chain immunoreceptor expression whereas the expression of γ-chain receptors is not affected. The low expression of ζ-chain immunoreceptors on the cell membrane of TCR⁺ cells is due to increased degradation. Similarly, the expression level of ζ-chain immunoreceptors in human T cells is significantly lower than those of γ-chain receptors. Low ζ receptor expression in peripheral T cells was due to the intracellular signaling domain and not the receptor's transmembrane region. Expression of both receptors decreased upon prolonged cultivation. Shortly after receptor engraftment, target cell lysis and induction of IFNY secretion is mediated with similar efficiency by both ζ- and γ-chain immunoreceptors. Upon prolonged propagation, however, ζ-chain immunoreceptor mediated T cell activation against tumor cells is more efficient indicating that the initial high expression level of γ-chain immunoreceptors compensates its lower cellular activation capacity.

Thus, although initially similar efficient, ζ-chain receptor grafted T cells are expected to be superior to γ-chain receptors with respect to achieve a long lasting anti-tumor response in vivo. On the other hand, the use of recombinant γ-chain immunoreceptors may limit an anti-tumor response much earlier preventing autoaggression of receptor grafted T cells. Under this point of view, the intracellular signaling domain can be utilized to fine-tune a recombinant receptor mediated immune response.

To summarize, the above findings prove that these recombinant immunoreceptors are useful for therapy, e.g., for adoptive immunotherapy.

Accordingly, in some embodiments, the present invention provides a recombinant immunoreceptor comprising (i) a first antigen binding segment comprising an scFv and (ii) a second segment linked to the first segment and comprising a transmembrane domain fused to an intracellular signalling domain selected from the group consisting of an FcεRIγ-chain derived transmembrane domain fused to a CD3ζ-chain derived intracellular signalling domain; a CD3ζ-chain derived transmembrane domain fused to a CD3ζ-chain derived intracellular signalling domain; a CD3ζ-chain derived transmembrane domain fused to an FcεRIγ-chain derived intracellular signalling domain; and a FcεRIγ-chain derived transmembrane domain fused to an FcεRIγ-chain derived intracellular signalling domain. In some embodiments, the first segment is specific for a viral antigen, synthetic antigen, tumor associated antigen, tumor specific antigen, mucosal antigen, superantigen, differentiation antigen, auto-immune antigen, self antigen, receptor ligand, cell bound growth factor, cell bound carbohydrate antigen or hapten. In further embodiments, the first segment is linked to the second segment via an extracellular spacer. In some preferred embodiments, the extracellular spacer is hIgG1-Fc. In still other embodiments, the first segment is specific for CEA, CA72-4 or CA19-9.

In further embodiments, the present invention provides a nucleic acid molecule encoding the foregoing recombinant immunoreceptors. In some embodiments, the present invention provides expression vector containing the nucleic acid molecules. In other embodiments, the present invention provides cells expressing the recombinant immunoreceptors. In some preferred embodiments, the cell is an immune cell. In still more preferred embodiments, the cell is a resting, activating or memory T lymphocyte, a cytotoxic lymphocyte (CTLs), a helper T cell, a non-T lymphocyte, a B cell, a plasma cell, a natural killer cell (NK), a monocyte, a macrophage, a granulocyte, an eosinophil cell or a dendritic cell. In some embodiments, the immune cell lacks endogenous CD3ζ

In still further embodiments, the present invention provides pharmaceutical composition containing the recombinant immunoreceptor described above, a nucleic acid molecule encoding said immunoreceptor, an expression vector comprising said nucleic acid molecule or an immune cell comprising said nucleic acid molecule.

In some embodiments, the present invention provides methods of preparing a composition for adoptive immunotherapy comprising combining with a pharmaceutically acceptable carrier the recombinant immunoreceptor described above, a nucleic acid molecule encoding said immunoreceptor, an expression vector comprising said nucleic acid molecule or an immune cell comprising said nucleic acid molecule.

In other embodiments, the present invention provides methods of treatment comprising providing a subject suffering from or at risk of suffering from a condition selected from the group consisting of cancer, infectious diseases, autoimmune diseases and graft rejection and administering to said subject the recombinant immunoreceptor as described above, a nucleic acid molecule encoding said immunoreceptor, an expression vector comprising said nucleic acid molecule or an immune cell comprising said nucleic acid molecule.

### BRIEF DESCRIPTION OF THE DRAWINGS

FIG. 1: Expression cassettes for the recombinant immunoreceptors used in this study

The numbers indicate the amino acids constituting the transmembrane (TM) and intracellular (IC) signaling domains of the immunoreceptor.

FIG. 2: Expression of endogenous CD3ζ in MD45 and MD27 T hybridoma cells

MD45 (A), MD27 (B) and peripheral human T cells (C) were intracellularly stained with the FITC-conjugated anti-ζ mAb G3 (Eshhar et al., Supra) with dual specificity for mouse and human CD3ζ (solid lines) or a FITC-conjugated, isotype-matched control mAb (BD Pharmingen, Freiburg, Germany) (thin lines). The cells were analyzed by flow cytometry and the histograms were overlayed.

FIG. 3: Expression of recombinant scFv-Fc-γ/γ and scFv-Fc-ζ/ζ receptors in TCR⁺ MD45 and TCR⁻ MD27 T cells

(A) MD45 and (B) MD27 T cells were grafted with the recombinant CC49-scFv-Fc-γ/γ or CC49-scFv-Fc-ζ/ζ immunoreceptor as described in Example 1. Non-transfected and CC49-scFv-Fc-γ/γ or CC49-scFv-Fc-ζ/ζ receptor grafted cells, respectively, were stained with FITC-conjugated control antibodies (dashed lines) or anti-murine CD3_{ε}, anti-TCRαβ, and receptor specific anti-human IgG1 antibodies (BD Pharningen, Freiburg, Germany), respectively (solid lines). Bound antibodies were detected by flow cytometry and fluorescence histograms were overlayed.

FIG. 4: Activation of TCR⁺ MD45 and TCR⁻ MD27 T cells grafted with the recomhinant scFv-Fc-γ/γ and scFv-Fc-ζ/ζ receptor, respectively

(A) MD45 and (B) MD27 T cells (5×10⁵ cells/well) were grafted with the recombinant CC49-scFv-Fc-γ/γ or CC49-scFv-Fc-ζ/ζ receptor, respectively, and incubated for 48 hrs in microtiter plates coated with anti-mouse CD3_{ε}mAb, anti-human IgG Fc mAb or anti-mouse TCRαβ mAb (each 10 μg/ml) (BD Pharmingen, Freiburg, Germany). IL-2 secreted by activated MD45 and MD27 T cells was detected by ELISA as described in Example 1.

FIG. 5: The recomhinant CC49-scFv-Fc-ζ/ζ immiinoreceptor is rapidly degraded in the presence of the endogenoiis TCR

The cell surface of viable MD45 (A) and MD27 (B) T cells (5×10⁷ cells each), grafted with the recombinant CC49-scFv-Fc-γ/γ or CC49-scFv-Fc-ζ/ζ receptor was biotinylated as described in Example 1. Biotinylated cells were cultured in medium at 37° C. and lysed 0, 2, 4, and 6 hrs after labeling (1×10⁷ cells each, 5×10⁷ cells/ml lysis buffer). The amount of biotinylated recombinant receptor was recorded by ELISA utilizing a plastic immobilized anti-human IgG Fc antibody for capture (10 μ/ml) and peroxidase conjugated streptavidin (1:10,000) for detection. The amount of biotinylated recombinant receptor in the cell lysate is given as percent initially present after biotinylation of grafted cells.

FIG. 6: Expression of recombinant receptors in peripiheral blood T cells

Peripheral blood T cells were retrovirally grafted with different scFv-Fc-γ/γ or scFv-Fc-ζ/ζ receptors (A-F), scFv-Fc-ζ/γ (H) or scFv-Fc-γ/ζ (K) receptors and scFv-Fc-CD28/CD28 (G) or scFv-Fc-CD28/CD28-ζ (J) immunoreceptors and stained simultaneously with PE-conjugated anti-CD3 and FITC-conjugated anti-human IgG Fc antibodies. For control, non-transduced (I) and cell that were transduced with an empty expression vector (L) were also stained with anti-CD3 and anti-human IgG antibodies, respectively. The cells were analyzed by flow cytometry and the data presented as dot blots.

FIG. 7: Stability of recombinant scFv-Fcγ/γ and scFv-Fc-ζ/ζ receptor expression in peripheral blood T cells

Peripheral blood T cells from two different healthy donors were retrovirally grafted with recombinant anti-CA19-9-receptors (NS19-9-scFv-Fc-γ/γ, NS19-9-scFv-Fc-ζ/ζ) or anti-CEA-receptors (BW431/26-scFv-Fc-γ/γ, BW431/26-scFv-Fc-ζ/ζ) and cultured for 37 days in the presence of 400 U/ml IL-2. Cells were harvested every third day, stained simultaneously with FITC-conjugated anti-CD3 and PE-conjugated anti-human IgG Fc antibodies and analyzed by flow cytometry.

(A-C) The number of cells with recombinant receptor expression [%] from total number of cells was recorded as described in Example 1.

(D-E) The mean red fluorescence of transduced and non-transduced CD3⁺ T cells.

(GI) The mean red fluorescence of transduced CD3⁺ T cells with detectable amounts of recombinant receptor expression on the cell surface.

FIG. 8: Antigen specific activation of T cells grafted with recombinant scFv-Fc-γ/γ and scFv-Fc-ζ/ζ receptors, respectively, upon prolonged cultivation

T cells from the peripheral blood were grafted with the BW431/26-scFv-Fc-γ/γ and BW431/26-scFv-Fc-ζ/ζ receptor, respectively, and propagated in the presence of 400 U/ml IL-2. At day 1 and at day 37 after receptor engraftment, T cells (0.625-5×10⁴ cells/well) were cocultivated with CEA LS174T and CEA A375 tumor cells (5×10⁴ cells/well), respectively.

(A) Viability of CEA⁺ LS174T target cells was determined calorimetrically by a tetrazolium salt based XTT-assay as described Example 1.

(B) IFN-γ secreted by receptor grafted T cells into the supernatant was determined by ELISA.

### DESCRIPTION OF THE INVENTION

The present invention relates to a recombinant immunoreceptor comprising (i) a first antigen binding segment comprising an scFv and (ii) a second segment linked to the first segment and comprising a transmembrane domain fused to an intracellular signaling domain, wherein
(a) the transmembrane domain comprises an FcεRIγ-chain derived transmembrane domain and the intracellular signaling domain comprises a CD3ζ-chain derived intracellular signaling domain;
(b) the transmembrane domain comprises a CD3ζ-chain derived transmembrane domain and the intracellular signaling domain comprises a CD3ζ-chain derived intracellular signaling domain;
(c) the transmembrane domain comprises a CD3ζ-chain derived transmembrane domain and the intracellular signaling domain comprises an FcεRIγ-chain derived intracellular signaling domain; or
(d) the transmembrane domain comprises a FcεRIγ-chain derived transmembrane domain and the intracellular signaling domain comprises an FcεRIγ-chain derived intracellular signaling domain.

The term “immunoreceptor” as used herein relates to any receptor which is capable of (a) binding to a desired antigen and (b) after binding of said antigen induce cellular activation. Based on the experiments of the examples, below, the person skilled in the art can construct nucleic acid molecules encoding such immunoreceptors according to standard methods of recombinant DNA technology. Preferably, the different segments of the immunoreceptor are derived from a human. Preferably, the intracellular signaling domain is an intracellular signaling domain with cell activation properties.

As used herein, the terms “CD3ζ-chain derived transmembrane domain, FcεRIγ-chain derived transmembrane domain, CD3ζ-chain derived intracellular signaling domain, FcεRIγ-chain derived intracellular signaling domain” relate to the corresponding domains of the CD3ζ-chain or FcεRIγ-chain having the same biological activity, i.e. membrane anchoring and signal transduction. These domains have (i) amino acid sequences corresponding to the naturally occurring amino acid sequences or (ii) amino acid sequences differing from the amino acid sequences of (i) by substitution(s), deletion(s) and/or substitution(s) of one or more amino acid sequences but have substantially the same biological function. These domains comprise the whole molecules or part of the molecules.

Amino acid sequences of the CD3ζ-chain and the FcεRIγ-chain and the nucleic acid sequences encoding said polypeptides are known to the person skilled in the art. Said nucleic acid sequences can be isolated from natural sources or can be synthesized according to known methods. For example, repertoires of T cell receptor segment encoding genes can be derived from natural sources such as peripheral blood lymphocytes (PBLs), tumor infiltrating T-cells or cloned cytotoxic T cells or cell lines, or can be derived from TCR V-gene segments created in part or completely synthetically. The scFv of the immunoreceptor of the present invention encoding gene can be cloned from immune T lymphocytes or from synthetic libraries such as phage display libraries, viral display libraries or others.

For the manipulation in prokaryotic cells by means of genetic engineering said nucleic acid sequences or parts of these sequences can be introduced into plasmids allowing a mutagenesis or a modification of a sequence by recombination of DNA sequences. By means of conventional methods (cf. Sambrook et al., 1989, Molecular Cloning: A Laboratory Manual, 2^{nd} edition, Cold Spring Harbor Laboratory Press, NY, USA) bases can be exchanged and natural or synthetic sequences can be added. In order to link the DNA fragments with each other adapters or linkers can be added to the fragments. Furthermore, manipulations can be performed that provide suitable cleavage sites or that remove superfluous DNA or cleavage sites. If insertions, deletions or substitutions are desired, in vitro mutagenesis, primer repair, restriction or ligation can be performed. As analysis methods usually sequence analysis, restriction analysis and other biochemical or molecular biological methods are used.

In a preferred embodiment, the first segment of the immunoreceptor of the present invention (i.e. scFv) is specific for a viral antigen, synthetic antigen, tumor associated antigen, tumor specific antigen, mucosal antigen, superantigen, differentiation antigen, auto-immune antigen, self antigen, receptor ligand, cell bound growth factor, cell bound carbohydrate antigen or hapten.

The data of the examples demonstrate that the intracellular signaling domain of recombinant immunoreceptors affects receptor expression dependent on the endogenous receptor repertoire of the grafted effector cell. Dependent on the intracellular signaling domain the recombinant receptor may require an additional extracellular spacer domain for stable expression in T cells that in turn also impacts receptor mediated cell activation. A rational design for the generation of recombinant immunoreceptors should, thus, address at least cell surface expression, that is dependent on the type of the grafted effector cell and the receptor's signaling domain, and sustained cell activation properties during prolonged propagation of receptor grafted cells. Thus, in a more preferred embodiment, the first segment of the recombinant immunoreceptor of the present invention is linked to the second segment via an extracellular spacer. Examples of suitable extracellular spacers are parts of (D8-molecules, hinge-CH₂/CH₃-domain of human/murine IgG1, hinge CH₂-domain of human/murine IgG1. Preferably, the extracellular spacer is hIgG1-Fc.

In an even more preferred embodiment, the first segment of the immunoreceptor of the present invention is specific for CEA, CA72-4, CA19-9 or other tumor-specific and tumor associated antigens, growth factors or viral antigens.

The present invention also relates to a nucleic molecule (genomic DNA, cDNA, RNA) encoding the recombinant immunoreceptor of the present invention. Preferably, the nucleic acid molecule encoding the immunoreceptor of the present invention receptor is inserted into a recombinant (expression) vector. Preferably, these vectors are plasmids, cosmids, viruses, bacteriophages and other vectors usually used in the field of genetic engineering. Vectors suitable for use in the present invention include, but are not limited to the T7-based expression vector for expression in bacteria, the pMSXND expression vector for expression in mammalian cells and baculovirus-derived vectors for expression in insect cells. Preferred vectors for transfection of immune cells are MMLV-derived retroviral vectors for expression in primary human leucocytes. Preferably, the nucleic acid molecule is operatively linked to the regulatory elements in the recombinant vector of the invention that guarantee the transcription and synthesis of an RNA in prokaryotic and/or eukaryotic cells that can be translated. The nucleotide sequence to be transcribed can be operably linked to a promoter like a T7, metallothionein I or polyhedrin promoter.

Preferred recombinant vectors usefuil for gene therapy are viral vectors, e.g. adenovirus, herpes virus, vaccinia, or, more preferably, an RNA virus such as a retrovirus. Even more preferably, the retroviral vector is a derivative of a murine or avian retrovirus. Examples of such retroviral vectors which can be used in the present invention are: Moloney murine leukemia virus (MoMuLV), Harvey murine sarcoma virus (HaMuSV), murine mammary tumor virus (NuMTV) and Rous sarcoma virus (RSV). Most preferably, a non-human primate retroviral vector is employed, such as the gibbon ape leukemia virus (GaLV), providing a broader host range compared to murine vectors.

Since recombinant retroviruses are defective, assistance is required in order to produce infectious particles. Such assistance can be provided, e.g., by using helper cell lines that contain plasmids encoding all of the structural genes of the retrovirus under the control of regulatory sequences within the LTR. Suitable helper cell lines are well known to those skilled in the art. Said vectors can additionally contain a gene encoding a selectable marker so that the transduced cells can be identified. Moreover, the retroviral vectors can be modified in such a way that they become target specific. This can be achieved, e.g., by inserting a polynucleotide encoding a sugar, a glycolipid, or a protein, preferably an antibody. Those skilled in the art know additional methods for generating target specific vectors. Further suitable vectors and methods for in vitro- or in vivo-gene therapy are described in the literature and are known to the persons skilled in the art; see, e.g., WO 94/29469 or WO 97/00957, Bromberg et al., Methods in Enzymology 346 (2002), 199-224.

Suitable host cells for expression are prokaryotic or eukaryotic cells, for example mammalian cells, bacterial cells, insect cells or yeast cells. The host cells of the invention are preferably characterized by the fact that the introduced nucleic acid molecule either is heterologous with regard to the transformed cell, i.e. that it does not naturally occur in these cells, or is localized at a place in the genome different from that of the corresponding naturally occurring sequence. These host cells include the E. coli strains HB101, DH1, x1776, JM101, JM109, BL21, XL1Blue and SG 13009, the yeast strain Saccharomyces cerevisiae and the animal cells L, A9, 3T3, FM3A, CHO, COS, Vero, HeLa and Hep3B. The transfection of these host cells (and immune cells) with the nucleic acid molecules of the present invention may be performed by any standard method including viral vectors, eukaryotic vectors and electrical or chemical means such as calcium phosphate transfection, dextrane sulphate transfection, liposomal transfection, electroporation etc.

Methods for the recombinant production of the receptor, derivatives, fragments etc. are well known to the person skilled in the art, e.g., an above described host cell is cultivated under conditions allowing the synthesis of the protein and the protein is subsequently isolated from the cultivated cells and/or the culture medium. Isolation and purification of the recombinantly produced receptor may be carried out by conventional means including preparative chromatography and affinity and immunological separations involving affinity chromatography with monoclonal or polyclonal antibodies.

The present invention also relates to an immune cell having a predefined biological specificity, wherein said immune cell is grafted with a recombinant immunoreceptor of the present invention, i.e. said immune cell is capable of expressing the recombinant immunoreceptor of the present invention in a fimctional form, i.e. the receptor is capable of (i) binding to the antigen and (ii) signal transduction. Various types of lymphocytes and non-lymphotic cells may be suitable, for example, a resting, activating or memory T lymphocyte, a cytotoxic lymphocyte (CTL), a helper T cell, a non-T lymphocyte, a B cell, a plasma cell, a natural killer cell (NK), a monocyte, a granulocyte, a macrophage, an eosinophil cell or a dendritic cell. Preferably, said immune cell is a human immune cell.

The present invention also relates to a pharmaceutical composition containing a recombinant receptor, nucleic acid molecule, expression vector or immune cell of the present invention. For administration the above compounds are preferably combined with suitable pharmaceutical carriers. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose.

The present invention allows the use of the immunoreceptor of the present invention or the modified immune cells in adoptive in vivo gene and/or immunotherapy, e.g., to combat cancer, virus infections, bacterial infections, parasitic infections or autoimmune diseases. Regarding organ or tissue graft or auto-reactive cells, the immunoreceptors reactive with TCR epitopes an host T cells which cause autoimmunity or graft rejection can be transfected in the host T cells for elimination of the reactive cells following in vivo transfer. For example, for immunotherapy T cells are isolated from a patient, transfected with a nucleic acid molecule or expression vector of the present invention and the transfected immune cells are re-administered to the patient in activated form.

For example, for adoptive immunotherapy T cells are isolated from the peripheral blood of tumor patients and grafted by a nucleic acid or expression vector according to the present invention and readministered to the patient.

The following Examples illustrate the invention.

### EXAMPLE 1

### Materials and Methods

(A) Cell Line, and Antibodies

MD45 is a mouse T cell hybridoma line, MD27 is a TCR_{α} deficient derivative therefrom (Eshhar et al., PNAS USA 1993; 90: 720-724), 293T cells are human embryonal kidney cells that express the SV40 large T antigen (Weijtens et al., Gene Ther. 199; 5: 1195-1203). LS174T (ATCC CCL 188) is a CEA-expressing colon carcinoma cell line, A375 (ATCC CRL 1619) is a melanoma cell line, OKT3 (ATCC CRL 8001) is a hybridoma cell line that produces the anti-CD3 mAb OKT3 (obtained from ATCC, Rockville, Md., USA). 293T cells were cultured in DME medium supplemented with 10% (v/v) FCS, all other cell lines were cultured in RPMI 1640 medium, 10% (v/v) FCS (all Life Technologies, Paisly, U.K.). Anti-CD3 mAb OKT3 was affinity purified from hybridoma supernatants utilizing a goat anti-mouse IgG2a antibody (Southern Biotechnology, Birmingham, Ala., USA) immobilized on sepharose (Amersham Pharmacia, Freiburg, Germany). The fluorescin-isothiocyanate (FITC)-conjugated anti-mouse TCRα/β (H57-597) and anti-mouse CD3_{ε}(7D169) mAbs and the anti-CD3ζ mAb G3 with specificity for both human and mouse derived CD3ζ, respectively, were purchased from Serotech, Oxford, UK. The phycoerythrin-(PE)-conjugated anti-CD3 mAb UCHTI was purchased from Dako, Hamburg, Germany. The goat anti-human IgG antibody and its FITC- and PE-conjugated F(ab′)₂ derivative were purchased from Southern Biotechnology, Birmingham, Ala., USA. The anti-human IFN-γ mAb NIB42, the anti-mouse IL-2 mAb JES6-1A12, the biotinylated anti-human IFN-γ and anti-mouse IL-2 mAbs 4S.B3 and JES6-5H4, respectively, were purchased from BD Pharmingen, San Diego, Calif., USA.

(B) Generation of Recombinant Immunoreceptors

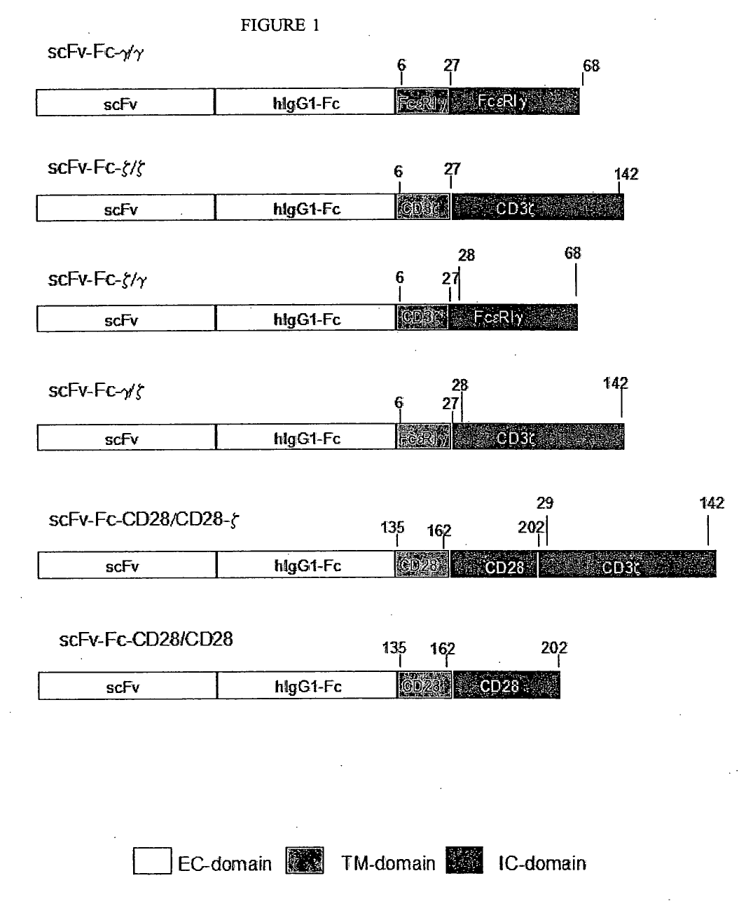

FIG. 1 and Table 1 summarize the recombinant immunoreceptors that were used for this study.

TABLE 1
Recombinant receptors used in this study.

| | | signalling | expression in^{a} | | | |
|---|---|---|---|---|---|---|
| scFv | | domain | MD45 | MD27 | 293T | CD3⁺ |
| (specificity) | recombinant receptor | (TM/IC) | (TCR⁺) | (TCR⁻) | (TCR⁻) | T cells |
| CC49 | CC49-scFv-Fc-γ/γ | γ/γ | + | + | + | + |
| (CA72-4) | CC49-scFv-Fc-ζ/ζ | ζ/ζ | + | + | + | + |
| | CC49-scFv-Fc-γ/ζ | γ/ζ | − | − | + | + |
| | CC49-scFv-Fc-ζ/γ | ζ/γ | − | − | + | + |
| NS19-9 | NS19-9-scFv-Fc-γ/γ | γ/γ | − | − | + | + |
| (CA19-9) | NS19-9-scFv-Fc-ζ/ζ | ζ/ζ | − | − | + | + |
| BW431/26 | BW431/26-scFv-Fc-γ/γ | γ/γ | − | − | + | + |
| (CEA) | BW431/26-scFv-Fc-ζ/ζ | ζ/ζ | − | − | + | + |
| | BW431/26-scFv-Fc-CD28/CD28 | CD28/CD28 | − | − | + | + |
| | BW431/26-scFv-Fc-CD28/CD28-ζ | CD28/CD28-ζ | − | − | + | + |

^{a}Recombinant immunoreceptors were expressed as described in Example 1.

The generation and expression of the CEA-specific BW431/26-scFv-Fc-γ/γ, BW431/26-scFv-Fc-ζ/ζ, BW431/26-scFv-Fc-CD28/CD28 and BW431/26-scFv-Fc-CD28/CD28-ζ receptors and of the CA72-4-specific CC49-scFv-Fc-γ/γ and CC49-scFv-Fc-ζ/ζ receptors were recently described in detail (Hombach et al., J. Immunol. 2001; 167: 1090-1096; Hombach et al., Int. J. Mol. Med. 1998; 2: 99-103; Hombach et al., Cancer Res. 2001; 61: 1976-1982; Hombach et al., J. Immunol. 2001; 167: 6123-6131; Hombach et al., Int. J. Cancer 2000; 88: 115-120). The anti-CA19-9-scFv was isolated from NS19-9 hybridoma cells by means of the recombinant phage antibody system (Amersham Biosciences, Freiburg, Germany). The resulting scFv-antibody retained specific antigen binding compared to the parental mAb NS19-9 mAb (unpublished data). To generate the retroviral expression cassettes for CA19-9-specific recombinant immunoreceptors, the NS19-scFv DNA was amplified by PCR and herewith flanked by NcoI (5′) and BglII (3′) restriction sites, respectively, utilizing the following set of oligonucleotide primers: 5′-CTA CGT ACC ATG GAT TTT CAG GTG CAG ATT TTC-3′(sense; SEQ ID NO:1) and 5′-GGT TCC AGC AGA TCT GGA TAC GGC-3′ (antisense, restriction sites are underlined; SEQ ID NO:2). The anti-CEA receptor DNA in pBullet (Weijtens et al., 2000; Hombach et al., Cancer Res. 2001; 61: 1976-1982) was cleaved by NcoI and BamHI and the BW431/26-scFv DNA was replaced by the digested NS19-9-scFv PCR product. The expression cassettes of the variant CC49-scFv-Fc-γ/ζ and CC49-scFv-Fc-ζ/γ immunoreceptors harboring either a γ-chain derived transmembrane region that was fused to the intracellular signalling domain of CD3ζ or vice versa a CD3ζ derived transmembrane domain fused to the intracellular signalling domain of the FcεRIγ chain were generated as follows: The cDNA coding for the intracellular signalling domain of CD3ζ and FcεRIγ, respectively, was PCR-amplified utilizing the following oligonucleotids comprising sequences for the transmembrane regions of CD3ζ and FcεRIγ and for BamHI and XhoI restriction sites, respectively: 5′-TACTGGATCCTCAGCTCTGCTATATCCTGGATGCCATCCTGTTTCTGTATGGAATTGT CCTCACCCTCCTCTACTGTAGAGTGAAGTTCAGCAGGAGCG-3′(γ/ζ-sense, SEQ ID NO:3), 5′- CTGCTACTCGAGGATTAGCGAGGGGGCAGGGC-3′(γ/ζ-antisense, SEQ ID NO:4), 5′TACTGGATCCCAAACTCTGCTACCTGCTGGATGGAATC CTCTTCATCTATGGTGTCATTCTCACTGCCTTGTTCCTGCGACTGAAGATCCAAGTGC GAAAG-3′(ζ/γ-sense, SEQ ID NO:5), 5′-CTGCTACTCGAGGAC TAAAGCTACTGTGGTGGTTTCTCATG-3 (ζ/γ-antisense; restriction sites are underlined, SEQ ID NO:6). Herewith, the sequences coding for the transmembrane regions of CD3ζ and FcεRIγ, respectively, were substituted with each other. The CC49-scFv-Fc-γ/γ receptor DNA in pBullet was cleaved by BamHI and XhoI, respectively and the γ/γ sequences were replaced by the digested γ/ζ and ζ/γ PCR product.

(C) Expression of Recombinant Immunoreceptors

Expression of the recombinant CC49-scFv-Fc-γ/γ and CC49-scFv-Fc-ζ/ζ receptors in MD45 cells was described elsewhere (Hombach et al., 1998). To express the recombinant CC49-scFv-Fc-γ/γ and CC49-scFv-Fc-ζ/ζ receptors in MD27 cells, plasmid DNA encoding the CC49-scFv-Fc-γ/γ and −ζ/ζ receptor DNA, respectively, was transfected into 2×10⁷ MD27 T cells by electroporation (one pulse, 250 V, 2400 μF) using the “gene pulse electroporator” (BioRad, Munich, FRG). After culture for two days, transfectants were selected in the presence of G418 (2 mg/ml; Gibco, Eggenheim, FRG). Oligoclonal cell populations that express the recombinant receptor were subcloned and used for further analysis. To express the recombinant receptors in T cells from the peripheral blood, the expression cassettes were inserted into the retroviral vector pBullet (Hombach et al., Cancer Res. 2001; 61: 1976-1982) as recently described (Hombach et al., J. Immunol. 2001; 167: 6123-6131). Retroviral transduction of peripheral T cells with recombinant receptors was described in detail elsewhere (Weijtens et al., 2000; Hombach et al., Cancer Res. 2001; 61: 1976-1982; Weijtens et al., Gene Ther. 1998; 5: 1195-1203) and receptor expression was monitored by flow cytometric analysis. Recombinant receptors were also expressed in 293T cells after transfection by calcium phosphate coprecipitation of 2×10⁶ 293T cells with 20 μg DNA of the retroviral expression vector. Cells were harvested after 48 hrs and subjected to analysis.

(D) Immunofluorescence Analysis

Recombinant immunoreceptors on 293T, MD45 and MD27 cells were detected by a FITC-conjugated F(ab′)₂ anti-human IgG1 antibody (2 μg/ml). Expression of murine CD3 and TCRαβ expression on MD45 and MD27 cells, respectively, was monitored utilizing the FITC-conjugated anti-mouse CD3_{ε} mAb 7D169 and the anti-mouse TCRαβ mAb H57-597 according the manufacturer's recommendations. Intracytoplasmatic expression of endogenous CD3ζ in MD45 and MD27 cells, respectively, was analyzed as follows: Briefly, MD45, MD27 and for control human peripheral T cells were fixed and permeabilized utilizing cytofix/cytoperm® solution (Pharmingen) according to the manufacturer's recommendations. After washing with PBS containing 1% (w/v) saponin (Pharmingen) the cells were incubated with 10 ill of a FITC-conjugated mouse anti-CD3ζ antibody (G3) with specificity for both human and mouse CD3ζ or an isotype matched control mAb. The cells were incubated for 30 min on ice, washed and analyzed by flow cytometry. T cells grafted with the recombinant receptor were identified by two color immunofluorescence utilizing a PE- or FITC-conjugated F(ab′)₂ anti-human IgG1 antibody (2 g/ml) and a FITC- or PE-conjugated anti-CD3 mAb (UCHT-1, 1:20). Immunofluorescence was analyzed using a FACScan™ cytofluorometer equipped with the CellQuest research software (Becton Dickinson, Mountain View, Calif.). To identify T cells with recombinant receptor expression, we set markers with 99% of non-transduced T cells beyond. The mean fluorescence intensity of CD3/recombinant receptor double positive T cells reflects the number of recombinant receptors expressed on the cell surface of grafted T cells.

(E) Biotin-Labeling of Transfected Mouse T Cells and Detection of Labeled Receptor Molecules

The cell surface of viable T hybridoma cells was labeled with biotin essentially as described (Ono et al., Immunity 1995; 2: 639-644). Briefly, 5×10⁷ transfected and non-transfected MD45 and MD27 T cells, respectively, were washed twice in cold PBS, pH 7.6, resuspended in 1 ml PBS, pH 7.6, and biotin-_{ε}-amidocaprone-acid-N-hydroxy-succinimid-ester (Sigma, Deisen-hofen, Germany) was added to a final concentration of 100 μ/ml. Cells were incubated for 1 h on ice and subsequently washed three times with RPMI 1640 medium, 10% (v/v) FCS. To monitor degradation of biotinylated recombinant receptors, transfected cells were incubated in RPMI 1640 medium, 10% (v/v) FCS at 37° C. and 5×10⁶ cells were lysed by addition of 1% (v/v) NP40 at each time point. Lysates were cleared by centrifugation and analyzed by ELISA for the presence of recombinant immunoreceptors utilizing a plastic bound anti-human IgG1 antibody (10 μg/ml) for capture and peroxidase-conjugated streptavidin (1:10,000) for detection. The reaction product was developed by ABTS® (Roche Diagnostics, Mannheim, Germany). The extinction of biotinylated, non-transfected MD45 and MD27 T cells, respectively, was subtracted and the values converted to percent of receptor initially present after biotinylation of grafted cells.

(F) Receptor Mediated Activation of Mouse MD45 and MD27 T Cells

Anti-mouse CD36ε, anti-mouse TCR-αβ and anti-human IgG1 antibodies (each 10 μg/ml) were coated onto 96 well microtiter plates. The plates were washed with PBS and transfected and non-transfected MD45 and MD27 cells (1×10⁵/well), respectively, were cultured in coated microtiter plates for 48 h at 37° C. The supernatants were harvested and the amount of secreted IL-2 was determined by ELISA with the solid phase rat anti-mouse IL-2 mAb JES6-1A12 (BD Biosciences, Freiburg, Germany) (2 μg/ml) and the biotinylated rat anti-mouse IL-2 mAb JES6-5H4 (BD Biosciences, Freiburg, Germany) (0.5 μg/ml). The reaction product was visualized by a peroxidase-streptavidin-conjugate (1:10,000) and ABTS®.

(G) Receptor mediated activation of grafted T cells from peripheral blood T cells (1.25×10⁴-10×10⁴/well), grafted with the anti-CEA-scFv-Fc-γ/γ and anti-CEA-scFv-Fc-ζ/ζ receptor, respectively, were cocultivated for 48 h in 96-well round bottom plates with CEA⁺ (LS174T) or CEA⁻ (A375) tumor cells (each 5×10⁴ cells/well). The culture supernatants were harvested and analyzed for secretion of IFN-γ by ELISA. Briefly, IFN-γ in the supernatant was bound to the solid phase anti-human IFN-γ mAb NIB42 (BD Biosciences, Freiburg, Germany) (1 μg/ml) and detected by the biotinylated anti-human IFN-γ mAb 4S.B3 (BD Biosciences, Freiburg, Germany) (0.5 μg/ml). The reaction product was visualized by a peroxidase-streptavidin-conjugate (1:10,000) and ABTS®. Specific cytotoxicity of receptor grafted T cells against target cells was monitored by a XTT based colorimetric assay as described previously (Jost et al., J. Immunol. Methods 1992; 147: 153-165). Briefly, receptor grafted and non-transduced T cells were cocultivated with CEA⁺ and CEA⁻ tumor cells as described above. After 48 hrs, XTT (2,3-bis(2-methoxy-4-nitro-5sulphonyl)-5[(phenyl-amino)carbonyl]-2H-tetrazolium hydroxide) reagent (1 mg/ml) (Cell Proliferation Kit II, Roche Diagnostics) was added to the cells and incubated for 30-90 min at 37° C. Reduction of XTT to formazan by viable tumor cells was monitored colorimetrically at an adsorbance wavelength of 450 nm and a reference wavelength of 650 nm. Maximal reduction of XTT was determined as the mean of 6 wells containing tumor cells only, the background as the mean of 6 wells containing RPMI 1640 medium, 10% (v/v) FCS. The non-specific formation of formazan due to the presence of effector cells was determined from triplicate wells containing effector cells in the same number as in the corresponding experimental wells. The number of viable tumor cells was calculated as follows:

$$\% viability = \frac{{OD}_{( exp . wells - corresponding number of effective cells )}}{{OD}_{( tumor cells without effectors - medium )}} \times 100$$

### EXAMPLE 2

### Expression of Recombinant γ- and ζ Receptors in the Presence of the Endogenous CD3/TCR Complex

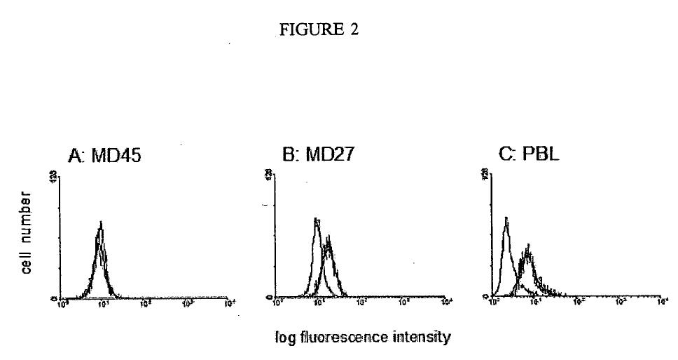

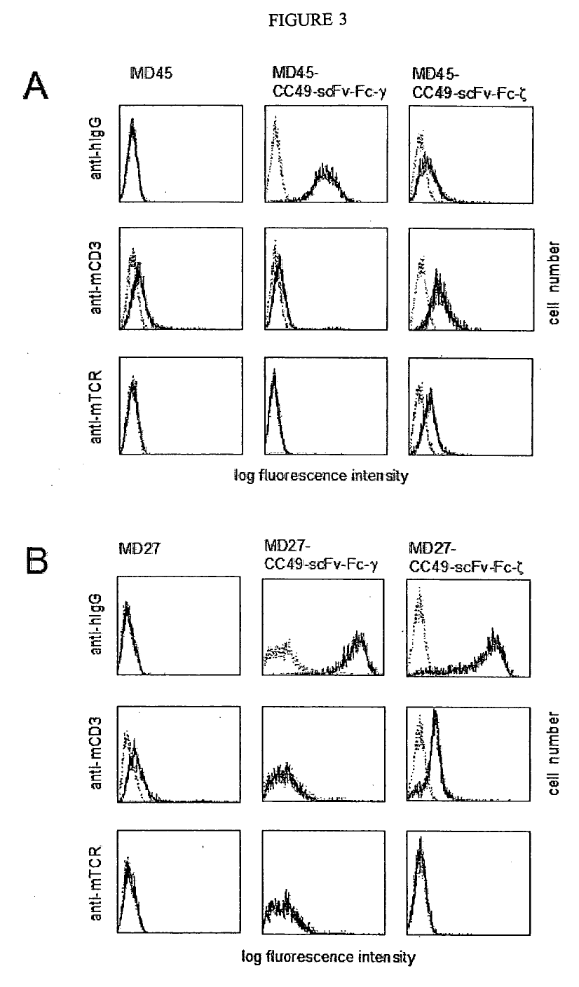

A panel of receptors that harbor similar extracellular antigen binding and spacer domains but different transmembrane and intracellular signalling domains derived either from CD3ζ, FcεRIγ or CD28 (FIG. 1) was generated. These receptors were expressed in several cell lines and primary T cells as summarized in Table 1. To analyze the expression of recombinant γ- and ζ-chain receptors in the presence of the endogenous TCR and, vice versa, their impact on CD3 and TCR expression the mouse CTL hybridoma cell lines MD45 and MD27 were stably transfected with plasmids coding for the recombinant CC49-scFv-Fc-γ/γ or CC49-scFv-Fc-ζ/ζ receptor, respectively, as described in Example 1. MD27 cells are a TCR⁻ derivative of MD45 cells lacking TCRα expression (Eshhar et al., 1993). MD45 and MD27 cell clones that stably express the CC49-scFv-Fc-γ/γ and CC49-scFv-Fc-ζ/ζ receptor, respectively, were isolated by limiting dilution techniques and expression of the recombinant receptor and of components of the endogenous CD3/TCR complex were recorded by FACS analysis utilizing representative cell clones. Non-transfected MD45 T cells, in contrast to TCR⁻ MD27 cells and human peripheral blood T cells, do not express detectable amounts of the endogenous ζ-chain as demonstrated by intracellular FACS analysis (FIG. 2). This is in accordance to observations that in T cell hybridomas the ζ-chain is synthesized in restricted amounts compared to other components of the CD3/TCR complex. Since the presence of a ζ-chain is essential for TCR assembly and cell surface expression we did not detect TCR expression and only record weak expression of CD3_{ε} on the cell surface of MD45 T cells (FIG. 3). Transfection of MD45 T cells with the CC49-scFv-Fc-ζ/ζ receptor, however, rescues expression of the endogenous TCR and enhances CD3_{ε} expression substantially. In contrast, expression of the recombinant CC49-scFv-Fc-γ/γ receptor in MD45 cells does not restore expression of the endogenous CD3/TCR complex (FIG. 3). Whereas expression of the CC49-scFv-Fc-ζ/ζ receptor in TCR⁻ MD27 cells results in enhanced CD3_{ε} expression, as expected, neither the recombinant ζ-chain nor the γ-chain immunoreceptor restores expression of the endogenous T cell receptor on the surface of TCR_{α} deficient MD27 cells. On the other hand the recombinant γ-chain receptor is expressed in a significant higher density on the cell membrane of MD45 T cells than the corresponding ζ-chain receptor whereas in TCR_{α} deficient MD27 cells recombinant γ- and ζ-chain receptors are both expressed with similar efficiency. This implies that the presence of a CD3/TCR complex on the cell membrane affects also the cell surface expression of recombinant ζ-chain receptors.

### EXAMPLE 3

### The Recombinant CC49-scFv-Fc-ζ/ζ Receptor Restores Signalling via the Endogenous CD3/TCR Complex in MD45 T Cells

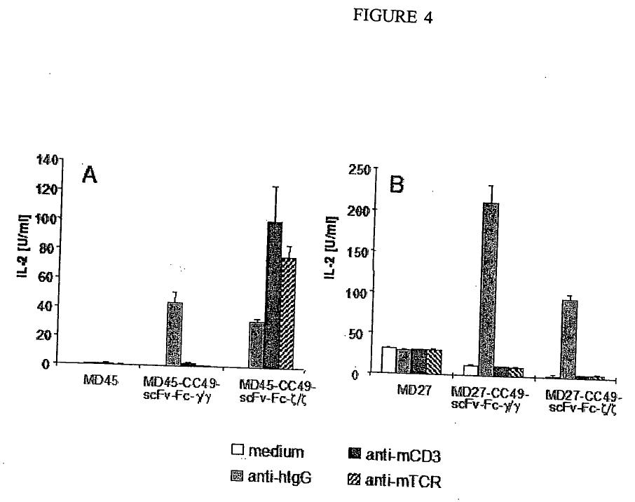

Specific signalling via recombinant γ-chain and ζ-chain receptors, respectively, triggers MD45 and MD27 T cells to secrete murine IL-2. Since the recombinant scFv-Fc-ζ/ζ-chain immunoreceptor restores expression of the endogenous CD3/TCR complex in MD45 T cells, it was asked whether this may also affect TCR mediated signalling. CC49-scFv-Fc-ζ/ζ and CC49-scFv-Fc-γ/γ transfected MD45 or MD27 T cells were stimulated with immobilized anti-human IgG, anti-mouse CD3_{ε} and anti-mouse TCRαβ antibodies, respectively, and IL-2 secretion was recorded. As demonstrated in FIG. 4, MD45 T cells that express the CC49-scFv-Fc-ζ/ζ receptor are efficiently activated to secrete IL-2 by crosslinking of the recombinant receptor and of the endogenous TCR/CD3 complex, respectively. In contrast, CC49-scFv-Fc-γ/γ receptor transfected MD45 T cells were activated only by crosslinking of the recombinant receptor but not by crosslinking of the endogenous TCR. MD27 cells that express the CC49-scFv-Fc-ζ/ζ and CC49-scFv-Fc-γ/γ receptor, respectively, were activated only upon crosslinking of the recombinant receptor but not of the endogenous CD3/TCR complex. Accordingly, the parental, non-transfected MD45 that do not express detectable amounts of an endogenous ζ-chain and TCR⁻ MD27 T cells were neither activated by immobilized antibodies directed against the endogenous CD3/TCR complex nor by an antibody directed against the human IgG domain of the recombinant receptor. These data indicate that the recombinant CC49-scFv-Fc-ζ/ζ receptor, in contrast to the γ-chain receptor, restores both expression and signalling by the endogenous CD3/TCR complex.

### EXAMPLE 4

### The Recombinant ζ Receptor is Rapidly Degraded in the Presence of TCRαβ

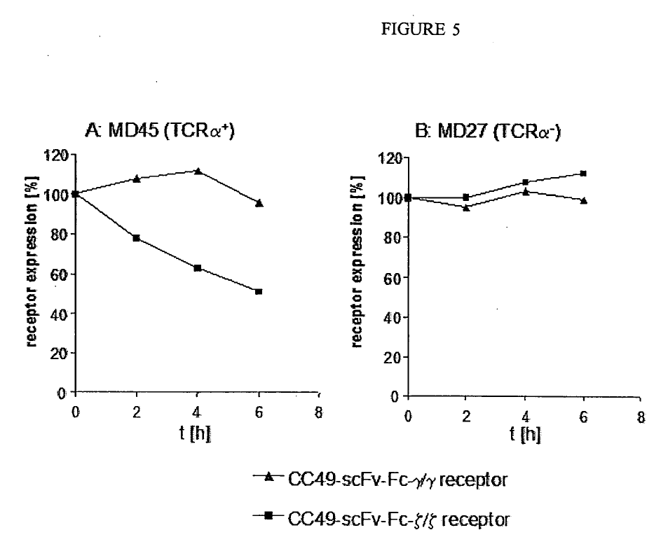

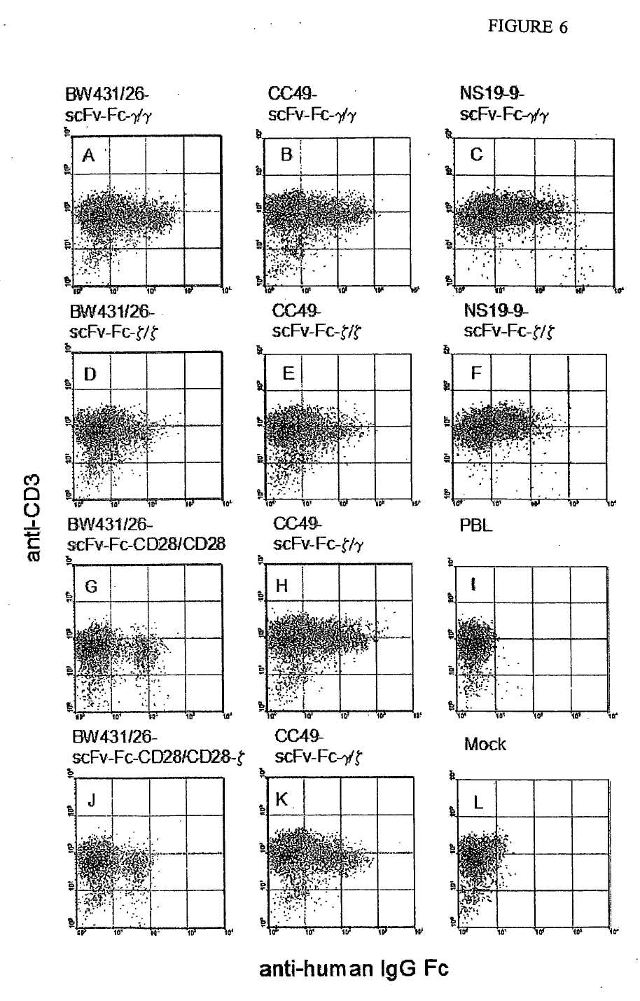

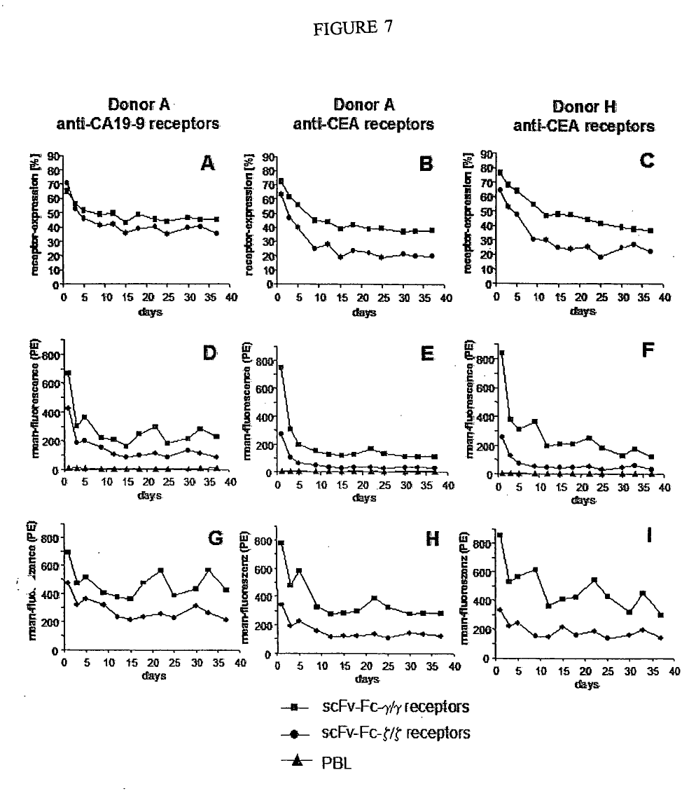

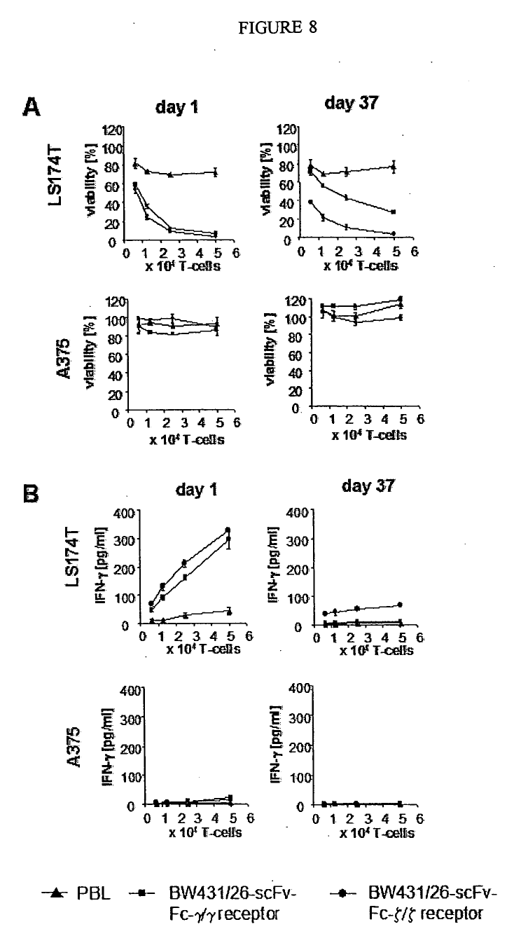

In presence of the endogenous TCRαβ, recombinant ζ-and γ-chain immunoreceptors are obviously differentially expressed on the cell surface with much lower expression levels of the ζ-chain receptor. It was therefore asked whether the presence or absence of the TCR affects the half-life time of recombinant γ- and ζ-chain receptors. CC49-scFv-Fc-γ/γ and CC49-scFv-Fc-ζ/ζ receptor grafted MD45 and MD27 T cells, respectively, were labeled with biotin. Biotin-labeled cell were cultured at 37° C., lysed at different time points and biotinylated receptor molecules were recorded as described in Example 1. In TCR⁺ MD45 T cells, the recombinant CC49-scFv-Fc-ζ/ζ receptor that restores TCR expression on the cell surface is rapidly degraded (50% of the receptor molecules present after 6 hrs) whereas the CC49-scFv-Fc-γ/γ receptor is expressed with a much longer half-life time (>90% of the receptor molecules present after 6 hrs) (FIG. 5A). In the absence of the endogenous TCR, however, both ζ- and γ-chain receptors are expressed in a similar fashion and with long half-life times (FIG. 5B). These data indicate that the half-life time of recombinant immunoreceptors with CD3ζ-derived signalling domain on the cell surface is substantially affected by expression of an endogenous TCR in the grafted cell.

### EXAMPLE 5

### Recombinant γ- and ζ-Chain Immunoreceptors are Expressed in a Different Fashion in Human Peripheral Blood T Cells

To analyze the impact of the signalling domain on the expression of recombinant receptors in human peripheral blood T cells and to dissect the role of the FcεRIγ vs. CD3ζ derived transmembrane domain, a panel of recombinant immunoreceptors was generated whose expression cassettes were inserted into the retroviral expression vector pBullet (Weijtens et al., 2000). These receptors harbor the same extracellular antigen binding and spacer domains with specificity for, tumor antigens (FIG. 1; Table 1). The transmembrane and intracellular domains, however, are composed of (i) transmembrane and intracellular domains that are both derived either from the FcεRIγ-(γ/γ) or CD3ζ-(ζ/ζ) chain, (ii) a FcεRIγ-chain derived transmembrane domain that is fused to the intracellular CD3ζ signalling domain (γ/ζ) and, vice versa, a CD3ζ-chain derived transmembrane domain that is fused to the intracellular FcεRIγ signalling domain (ζ/γ), respectively, and (iii) a CD28 derived transmembrane and intracellular signalling domain with (CD28/CD28-ζ) or without (CD28/CD28) the intracellular CD3ζ signalling domain (FIG. 1; Table 1). Peripheral blood T cells from healthy donors were grafted with this panel of recombinant immunoreceptors and the level of receptor expression was recorded by FACS analysis. As exemplarily demonstrated in FIG. 6 and summarized in Table 2, retroviral transduction of peripheral blood lymphocytes resulted in highly efficient expression of all immunoreceptors. Noteworthy, the recombinant scFv-Fc-γ/γ receptors (FIG. 6A-C; Table 2) appeared to be expressed in higher densities on the cell surface of peripheral blood T cells than the homologous scFv-Fc-ζ/ζ chain receptors (FIG. 6D-F; Table 2). In contrast, all receptors were expressed in similar densities on the cell surface of TCR⁻ human 293T cells (datas not shown).

TABLE 2
Expression of recombinant receptors in peripheral T cells

| ^{(A)}EC^{a} | CC49-scFv-Fc | | | | NS19-9-scFv-Fc | | BW431/26-scFv-Fc | | | |
|---|---|---|---|---|---|---|---|---|---|---|
| ^{(B)}TM/IC^{b} | \tilde{γ}/γ | ζ/ζ | ζ/γ | γ/ζ | γ/γ | ζ/ζ | γ/γ | ζ/ζ | CD28/ | CD28/ |
| | | | | | | | | | CD28-ζ | CD28 |
| ^{(C)}Donor^{c} | mean-fluorescence^{d} | | | | | | | | | |
| 1 | n.d.^{e} | n.d. | n.d. | n.d. | 105.80 | 71.86 | 99.65 | 47.53 | n.d. | n.d. |
| 2 | 176.55 | 97.52 | 179.59 | 88.34 | n.d. | n.d. | 151.22 | 64.79 | n.d. | n.d. |
| 3 | 131.39 | 67.98 | 128.06 | 78.88 | n.d. | n.d. | 118.46 | 59.54 | n.d. | n.d. |
| 4 | 122.8 | 84.07 | 108.73 | 95.69 | n.d. | n.d. | 82.94 | 50.72 | n.d. | n.d. |
| 5 | n.d. | n.d. | n.d. | n.d. | 375.69 | 132.57 | 243.15 | 87.96 | n.d. | n.d. |
| 6 | 127.52 | 85.61 | n.d. | n.d. | 125.2 | 71.14 | 127.13 | 64.83 | n.d. | n.d. |
| 7 | n.d. | n.d. | n.d. | n.d. | 127.43 | 92.3 | n.d. | n.d. | n.d. | n.d. |
| 8 | n.d. | n.d. | n.d. | n.d. | n.d. | n.d. | 74.28 | 53.37 | 54.14 | 92.38 |
| 9 | n.d. | n.d. | n.d. | n.d. | 76.0 | 55.28 | 102.83 | 46.92 | 61.35 | 84.92 |
| 10 | n.d. | n.d. | n.d. | n.d. | n.d. | n.d. | 64.43 | 40.93 | 46.84 | 76.75 |
| 11 | n.d. | n.d. | n.d. | n.d. | 101.1 | 79.52 | n.d. | n.d. | n.d. | n.d. |
| 12 | n.d. | n.d. | n.d. | n.d. | 144.86 | 90.54 | 116.02 | 79.02 | n.d. | n.d. |
| 13 | n.d. | n.d. | n.d. | n.d. | 132.95 | 57.79 | 178.21 | 50.93 | n.d. | n.d. |
| n | 4 | 4 | 3 | 3 | 7 | 7 | 11 | 11 | 3 | 3 |
| mean | 139.565 | 83.8 | 138.79 | 87.64 | 116.19 | 74.06 | 123.48 | 58.78 | 54.11 | 84.68 |
| (SD) | (21.57) | (10.51) | (29.91) | (6.88) | (21.58) | (13.44) | (49.29) | (13.74) | (5.92) | (6.38) |

^{a}Extracellular scFv-domains with specificity for the CA72-4 (CC49-scFv), CA19-9 (NS19-9-scFv) and CEA (BW431/26-scFv) tumor antigens.
^{b}Transmembrane (TM) and intracellular (IC) signalling domain.
^{c}T cells of healthy donors were engrafted with the recombinant receptor as described in Example 1.

It was asked whether the observed differences are due to the intracellular signalling domains of CD3ζ and FcεRIγ, respectively, or their transmembrane regions that are highly homologous. As demonstrated in FIG. 6H,K and Table 2, the recombinant scFv-Fc-γ/ζ receptor is expressed on the cell surface of grafted T cells in a lower density than the corresponding scFv-Fc-ζ/γ receptor indicating that the different expression patterns of recombinant receptors results rather from the intracellular signalling moiety than the transmembrane domain of the receptor. This is further substantiated by the observation that engraftment of an intracellular CD3ζ signalling domain to a recombinant immunoreceptor that harbors a CD28 derived transmembrane and intracellular signalling domain (Hombach et al., Cancer Res. 2001; 61:1976-1982) also substantially impairs the level of receptor expression (FIG. 6G,J; Table 2).

### EXAMPLE 6

### Correlation of Recombinant scFv-Fc-γ/γ and scFv-Fc-ζ/ζ Receptor Expression with Receptor Mediated T Cell Activation

T cells from the peripheral blood of two different donors were grafted with two different sets of homologous scFv-Fc-γ/γ and scFv-Fc-ζ/ζ receptors, respectively, and the number of CD3⁺ T cells with recombinant receptor expression was continuously monitored by flow cytometry over 37 days. Receptor expression was monitored utilizing a PE-conjugated anti-human IgG antibody that is much more sensitive for detection than the FITC-conjugated, homologous antibody (data not shown). T cells were regarded positive for recombinant receptor expression at a cut off value that was defined utilizing non-transduced T cells with >99% of these cells beyond this value. Herewith, nearly homogenous T cell populations with initial expression rates of recombinant γ- and ζ-chain receptors between 60-80% of the total population were obtained. The number of T cells with receptor expression was recorded and, as an indicator for the density of recombinant receptor expression on the cell surface, also the red mean fluorescence of the whole cell population and of those cells above the cut off value. As summarized in FIG. 7, the number of T cells with detectable amounts of recombinant γ- or ζ-chain immunoreceptors decreased from initially similar numbers and remained constant after day 5-10 (FIG. 7A-C). Compared to scFv-Fc-γ/γ receptor grafted T cells, however, the number of T cells with detectable amounts of scFv-Fc-ζ/ζ receptors decreased to substantially lower numbers. Concomitantly, the density of both recombinant γ- and ζ-chain immunoreceptors on the cell surface also decreased over the first 5-10 days of cultivation; the expression of the ζ-chain receptor, however, stabilized at a lower level than those of the homologous γ-chain receptor (FIG. 7D-F). This phenomenon is unlikely to be due to different growth kinetics of transduced and non-transduced T cells because the density of the recombinant receptor on the cell surface of those T cells that were gated for detectable receptor expression decreased in a similar fashion (FIG. 7G-I).

To compare the efficiency of cellular activation mediated by γ- and ζ-chain receptors early after retroviral engraftment vs. at day 37 of continuous cell culture, specific target cell lysis and IFN-γ secretion of anti-CEA receptor grafted T cells from donor H were recorded (FIG. 7C,F,I). Initially, recombinant γ-chain (BW431/26-scFv-Fc-γ/γ) and ζ-chain (BW431/26-scFv-Fc-ζ/ζ) receptor grafted T cells lysed CEA⁺ tumor cells with similar efficiency. Upon prolonged cultivation, however, target cell lysis by T cells grafted with the γ-chain receptor was substantially reduced whereas lysis by ζ-chain receptor grafted T cells was not altered (FIG. 8A). Accordingly, both γ- and ζ-chain receptor grafted T cells secreted initially high amounts of IFN-γ upon cocultivation with CEA⁺ tumor cells. After propagation for 37 days, however, γ-chain receptor grafted T cells did no more secrete detectable amounts of IFN-γ upon cocultivation with CEA⁺ tumor cells whereas ζ-chain receptor grafted T cells still secreted detectable amounts of IFN-γ, although at low levels (FIG. 8B). These findings are noteworthy taking into account that initially the number of T cells that express the recombinant ζ-chain receptor was only slightly lower than the number of T cells with γ-chain receptor expression (about 65% vs. 80%) (FIG. 7C). Upon prolonged cultivation, however, the number of T cells with detectable amounts of the ζ-chain receptor decreased to about 20% whereas the number of T cells equipped with the γ-chain receptor was still about 40% (FIG. 7C). Taken together, recombinant γ-chain receptor mediated T cell activation against antigen-positive cells is initially similar efficient than those mediated by the ζ-chain receptor, but becomes less effective upon prolonged cultivation of receptor grafted T cells despite still more stable expression than the ζ-chain receptor.

1. A recombinant immunoreceptor comprising (i) a first antigen binding segment comprising an scFv and (ii) a second segment linked to the first segment and comprising a transmembrane domain fused to an intracellular signaling domain, said second segment selected from the group consisting of an FcεRIγ-chain derived transmembrane domain fused to a CD3ζ-chain derived intracellular signaling domain; a CD3ζ-chain derived transmembrane domain fused to a CD3ζ-chain derived intracellular signaling domain; a CD3ζ-chain derived transmembrane domain fused to an FcεRIγ-chain derived intracellular signaling domain; and a FcεRIγ-chain derived transmembrane domain fused to an FcεRIγ-chain derived intracellular signaling domain.

2. The recombinant immunoreceptor of claim 1, wherein the first segment is specific for a viral antigen, synthetic antigen, tumor associated antigen, tumor specific antigen, mucosal antigen, superantigen, differentiation antigen, auto-immune antigen, self antigen, receptor ligand, cell bound growth factor, cell bound carbohydrate antigen or hapten.

3. The recombinant immunoreceptor of claim l, wherein the first segment is linked to the second segment via an extracellular spacer.

4. The recombinant immunoreceptor of claim 3, wherein the extracellular spacer is hIgG1-Fc.

5. The recombinant immunoreceptor of claim 1, wherein the first segment is specific for CEA, CA72-4 or CA19-9.

6. A nucleic acid molecule encoding the recombinant immunoreceptor of claim 1.

7. An expression vector containing the nucleic acid molecule of claim 6.

8. A cell expressing the recombinant immunoreceptor of claim 1.

9. The cell of claim 8, which is an immune cell.

10. The immune cell of claim 9, which is selected from the group consisting of a resting, activating or memory T lymphocyte, a cytotoxic lymphocyte (CTLs), a helper T cell, a non-T lymphocyte, a B cell, a plasma cell, a natural killer cell (NK), a monocyte, a macrophage, a granulocyte, an eosinophil cell and a dendritic cell.

11. The immune cell of claim 10, wherein said immune cell lacks endogenous CD3ζ

12. A pharmaceutical composition containing the recombinant immunoreceptor of claim 1, a nucleic acid molecule encoding said immunoreceptor, an expression vector comprising said nucleic acid molecule or an immune cell comprising said nucleic acid molecule.

13. A method of preparing a composition for adoptive immunotherapy comprising combining with a pharmaceutically acceptable carrier the recombinant immunoreceptor of claim 1, a nucleic acid molecule encoding said immunoreceptor, an expression vector comprising said nucleic acid molecule or an immune cell comprising said nucleic acid molecule.

14. A method of treatment comprising providing a subject suffering from or at risk of suffering from a condition selected from the group consisting of cancer, infectious diseases, autoimmune diseases and graft rejection and administering to said subject the recombinant immunoreceptor of claim 1, a nucleic acid molecule encoding said immunoreceptor, an expression vector comprising said nucleic acid molecule or an immune cell comprising said nucleic acid molecule.
